(19) United States
(12) Patent Application Publication
(10) Pub. No.: US 2017/0112659 A1
(43) Pub. Date: Apr. 27, 2017

(54) ORAL CAVITY INSERTION TOOL

(71) Applicant: FFC CORPORATION, Nagasaki-shi, Nagasaki (JP)

(72) Inventor: Yoshiaki Akihiro, Musashino-shi, Tokyo (JP)

(73) Assignee: FFC CORPORATION, Nagasaki-shi, Nagasaki (JP)

(21) Appl. No.: 14/894,735

(22) PCT Filed: Jun. 22, 2015
(86) PCT No.: PCT/JP2015/067829

§ 371 Date: Nov. 30, 2015

(30) Foreign Application Priority Data
Jun. 3, 2015 (JP) 2015-113219

(51) Int. Cl. A61F 5/56; A61M 16/04

(52) U.S. Cl. CPC A61F 5/566; A61M 16/049

(57) ABSTRACT

An oral cavity insertion tool (1) has a flat base part (10), which includes a pressing piece (30) that is tabular and folded back forward at the upper end, a pressing piece (40) that is tabular and folded back forward at the lower end and, and an air hole (13) that penetrates in forward and backward directions, a backward projection part, which includes an opposing wall part (21) that projects backward from the center of the base part (10), provides a gap in which upper teeth can be inserted between the opposing wall part (21) and the base part (10), and faces the base part (10), and an opposing wall part (81) that provides a gap in which lower teeth can be inserted between the opposing wall part (81) and the base part (10), and faces the base part (10), and a lingual frenotomy pressing plate (50), which is tabular and provided to extend downward and backward from the rear end of the backward projection part, and the lingual frenotomy pressing plate (50) has a pressing part (51) that curves to curl up, towards the back, at the rear end.

### TECHNICAL FIELD

The present invention relates to an oral cavity insertion tool that is inserted in the oral cavity to secure the respiratory tract.

### BACKGROUND ART

Heretofore, many people of all ages and sex have suffered the symptoms of sleep apnea syndrome. The snoring and apnea condition is a symptom of this sleep apnea syndrome, and is caused when the respiratory tract is closed during sleep due to the short distance from the mental spine to the lingual frenotomy in the oral cavity. Also, sleep apnea syndrome may become a risk factor of lifestyle diseases. Besides sleep apnea syndrome, there are cases where orders from the brain cease due to fainting and so on and the relaxed respiratory muscle structure influences to close the respiratory tract.

Against such closure of the respiratory tract, in clinical therapy, constriction of the respiratory tract, which occurs when the tongue root sinks in the respiratory tract, is prevented by shifting the lower jaw forward to assume reversed occlusion, in which the lower jaw is placed ahead of the upper jaw, and pushing forward the lingual frenotomy for a distance to match the distance the lower jaw is shifted forward, and the respiratory tract is secured.

Patent literature 1 discloses an oral cavity insertion tool that is inserted in the oral cavity to improve the symptoms of sleep apnea syndrome. According to patent literature 1, reversed occlusion is assumed by guiding the front teeth in the lower jaw forward and leading the lower jaw forward with a tongue-like guide plate, and also the tongue is held in a forward posture by pressing the lingual frenotomy or the mucous membrane of the tongue base part with the guide plate. According to patent literature 1, the respiratory tract is secured by pressing the lingual frenotomy or the mucous membrane of the tongue base part with the guide plate and holding the tongue in a forward posture, in addition to assuming reversed occlusion.

Here, the distance from the mental spine to the lingual frenotomy varies with age, sex and so on. For example, generally speaking, women or young children show a shorter distance from the mental spine to the lingual frenotomy than adult males do.

### CITATION LIST

### Patent Literature

Patent Literature 1: Japanese Patent Application Publication No. 2005-95218

### SUMMARY OF INVENTION

### Technical Problem

However, referring to patent literature 1, since the distance from the mental spine to the lingual frenotomy varies per user of the oral cavity insertion tool, there are problems that, when the oral cavity insertion tool is inserted in the oral cavity, the lingual frenotomy or the mucous membrane of the tongue base part cannot be pressed sufficiently, or the force by which the guide plate presses the lingual frenotomy or the mucous membrane of the tongue base part is too strong causes pain.

It is therefore an object of the present invention to provide an oral cavity insertion tool that can reliably press the lingual frenotomy or the mucous membrane of the tongue base part, without causing pain, and secure the respiratory tract, regardless of the distance from the mental spine to the lingual frenotomy.

### Solution to Problem

According to a first aspect, the oral cavity insertion tool of the present invention provides an oral cavity insertion tool that is wholly elastically deformable, that is inserted in the oral cavity, and that has a flat base part, which includes a first pressing piece that is tabular and folded back forward at the upper end, a second pressing piece that is tabular and folded back forward at the lower end, and an air hole that penetrates in forward and backward directions, a backward projection part that projects backward from the center of the base part, which includes a first opposing wall part that provides a gap in which upper teeth can be inserted between the first opposing wall part and the base part, and faces the base part, and a second opposing wall part that provides a gap in which lower teeth can be inserted between the second opposing wall part and the base part, and faces the base part, and a lingual frenotomy pressing plate that is tabular and provided to extend downward and backward from the rear end of the backward projection part, and the lingual frenotomy pressing plate has a pressing part that curves to curl up, towards the back, at the rear end.

In addition to the above first aspect, according to a second aspect, the oral cavity insertion tool of the present invention further has a forward projection part that projects forward from the center of the base part, which includes a third opposing wall that provides a gap in which the upper lip can be inserted between the third opposing wall and the base part, and faces the base part, and a fourth opposing wall that provides a gap in which the lower lip can be inserted between fourth opposing wall and the base part, and faces the base part.

In addition to the above second aspect, according to a third aspect, in the oral cavity insertion tool of the present invention, the forward projection part is provided around the air hole, and includes a projecting part that projects in the air hole when seen from the front.

In addition to the above first aspect, according to a fourth aspect, in the oral cavity insertion tool of the present invention, the pressing part includes an erecting limb that is provided above, along the forward and backward directions.

In addition to the above first aspect, according to a fifth aspect, the oral cavity insertion tool of the present invention further has a dividing wall that is provided farther in the back than the air hole, and provided to divide the air hole when seen from the front, and the dividing wall includes a projecting wall that projects in the air hole when seen from the front.

In addition to the above first aspect, according to a sixth aspect, the oral cavity insertion tool of the present invention further has a replacement member that has the second opposing wall part and the lingual frenotomy pressing plate, abuts the base part, and is detachably attached to the base part, the base part having a different plate width in the portion where the replacement member abuts.

By inserting the oral cavity insertion tool in the oral cavity in the state in which the base part abuts teeth and gums, inserting upper teeth between the first opposing wall part and the base part, inserting lower teeth between the second opposing wall part and the base part, and, furthermore, by pressing the lingual frenotomy or the mucous membrane of the tongue base part with the pressing part of the lingual frenotomy pressing plate, the pressing part is pressed forward by the lingual frenotomy or the mucous membrane of the tongue base part and deforms elastically to curl upward, so as to ensure that those who use the oral cavity insertion tool, including women, young children and so on who exhibit a short distance from the mental spine to the lingual frenotomy compared to adult males and so on, feel no pain, and, for adult males and so on who exhibit a long distance from the mental spine to the lingual frenotomy compared to women, young children and so on, the pressing part abuts the lingual frenotomy or the mucous membrane of the tongue base part, and the pressing part reliably presses the lingual frenotomy or the mucous membrane of the tongue base part.

### Advantageous Effects of Invention

According to the present invention, it is possible to reliably press the lingual frenotomy, without causing pain, and secure the respiratory tract, regardless of the distance from the mental spine to the lingual frenotomy.

### BRIEF DESCRIPTION OF DRAWINGS

FIG. 1 is a rear view of an oral cavity insertion tool according to an embodiment of the present invention;

FIG. 2 is a front view of an oral cavity insertion tool according to an embodiment of the present invention;

FIG. 3 is a plan view of an oral cavity insertion tool according to an embodiment of the present invention;

FIG. 4 is an A-A cross-sectional view of FIG. 1;

FIG. 5 is an enlarged side view of a tongue pressing part according to an embodiment of the present invention;

FIG. 6 is a side view of a replacement member according to an embodiment of the present invention;

FIG. 7 is a bottom view of a replacement member according to an embodiment of the present invention;

FIG. 8 is a cross-sectional view of an oral cavity insertion tool according to an embodiment of the present invention, with a base part having a different plate width;

FIG. 9 is a cross-sectional view of a state in which an oral cavity insertion tool according to an embodiment of the present invention is inserted in the oral cavity; and

FIG. 10 is a cross-sectional view of a state in which an oral cavity insertion tool according to an embodiment of the present invention, with a base part of a different plate width, is inserted in the oral cavity.

### DESCRIPTION OF EMBODIMENTS

Now, an oral cavity insertion tool according to an embodiment of the present invention will be described below, in detail, with reference to the accompanying drawings. In the drawings, the x axis, the y axis and the z axis constitute a triaxial rectangular coordinate system, which will be described on the premise that the positive direction of the y axis is the forward direction, the negative direction of the y axis is the backward direction, the x axis directions are the left and right directions, the positive direction of the z axis is the upward direction, and the negative direction of the z axis is the downward direction.

<Structure of Oral Cavity Insertion Tool>

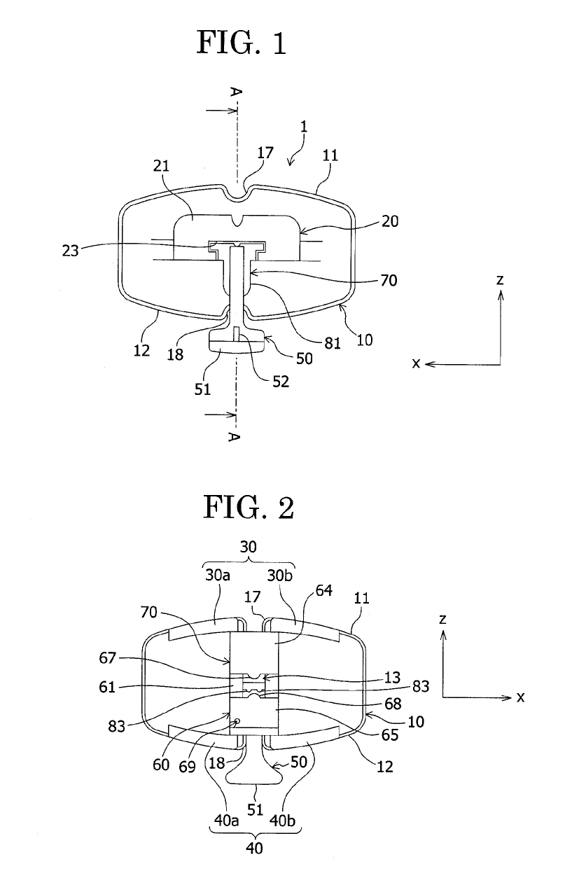

An oral cavity insertion tool 1 according to an embodiment of the present invention will be described below, in detail, with reference to FIG. 1 to FIG. 7.

The oral cavity insertion tool 1 is formed in one piece with elastic synthetic resin, and wholly elastically deformable. The synthetic resin to form the oral cavity insertion tool 1 is typically polyester elastomer, silicone rubber, polyethylene vinyl chloride and/or the like.

The oral cavity insertion tool 1 has a base part 10, a rear support part 20, a pressing piece 30, a pressing piece 40 and a replacement member 70.

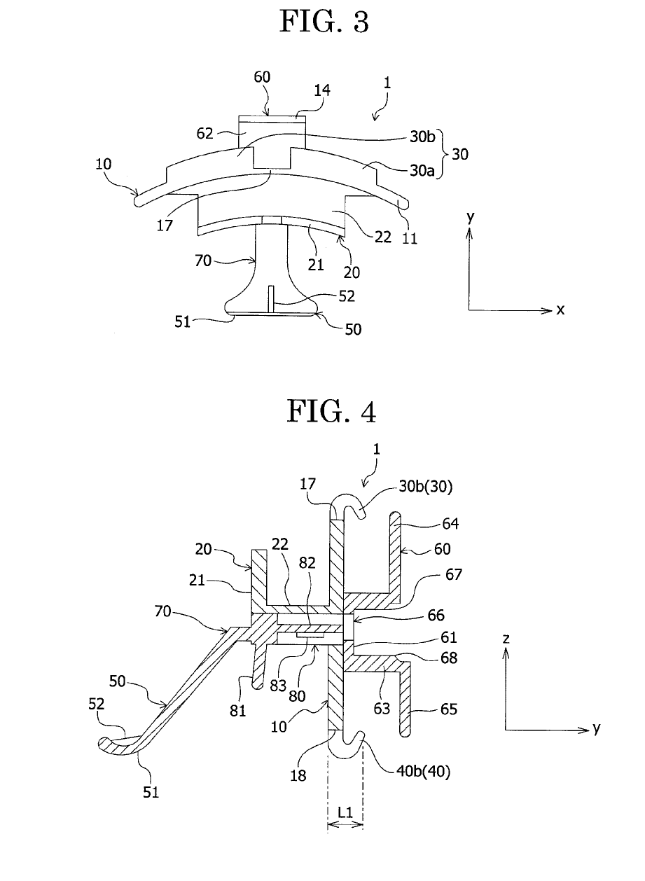

The base part 10 is flat, and has a curved shape that, as shown in FIG. 3, gradually projects forward from the left and right end parts to the center part. In the center of the base part 10, an air hole 13 that penetrates in the forward and backward directions, which correspond to the direction of the plate width, is provided. In the upper end 11 of the base part 10, a notch part 17, which is cut inward, is provided between a pair of a pressing piece 30a and a pressing piece 30b. In the lower end 12 of the base part 10, which is the opposite side of the upper end 11, a notch part 18, which is cut inward, is provided between a pair of a pressing piece 40a and a pressing piece 40b.

The rear support part 20 is formed in one piece with the base part 10, and provided to project backward in the center of the base part 10. In the rear support part 20, an erecting part 22, which is provided behind the center of base part 10, and an opposing wall part 21, which is a first opposing wall part to face the base part 10, which is provided to extend upward from the rear end of the erecting part 22, and which provides a gap in which upper teeth can be inserted, between the opposing wall part 21 and the base part 10, are formed. In the rear support part 20, a concave part 23, which is provided to extend from the front end to the rear end to communicate with the air hole 13, is formed (see FIG. 1).

The pressing piece 30 is flat and tabular, formed as one piece with the base part 10, and folds back forward at the upper end 11 of the base part 10. The pressing piece 30 is formed with the pair of the pressing piece 30a and the pressing piece 30b, which are formed at a predetermined gap at the upper end 11 of the base part 10.

A pressing piece 40 is flat and tabular, formed as one piece with the base part 10, and folded back forward at the lower end 12 of the base part 10. The pressing piece 40 is formed with a pair of a pressing piece 40a and a pressing piece 40b, which are formed at a predetermined gap at the lower end 12 of the base part 10.

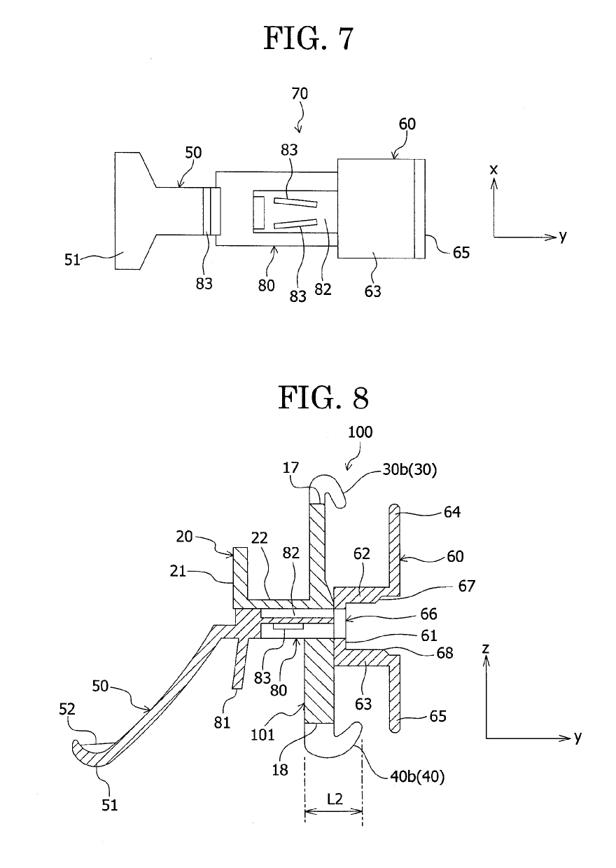

The replacement member 70 is detachably attached to the base part 10. The replacement member 70 has a lingual frenotomy pressing plate 50, a forward projection part 60, and a middle part 80, as shown in FIG. 6 and FIG. 7.

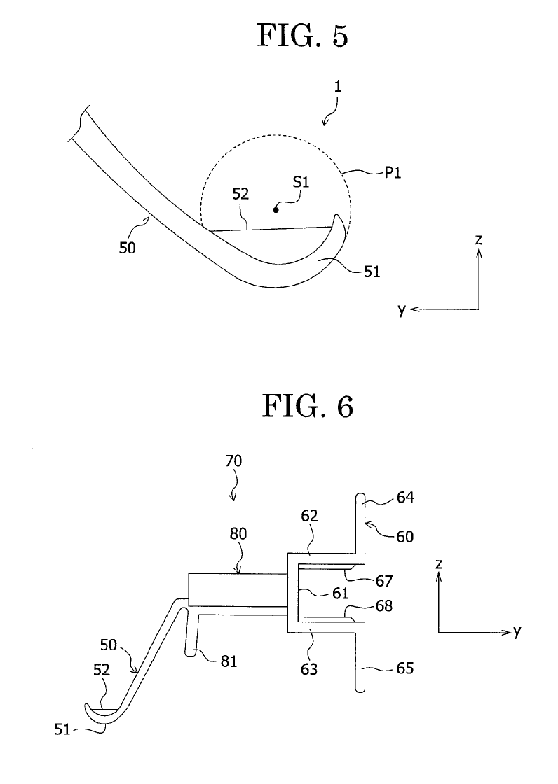

The lingual frenotomy pressing plate 50 is tabular, and provided to extend backward and downward from the rear end of the middle part 80. At the rear end of the lingual frenotomy pressing plate 50, a pressing part 51 is provided. As shown in FIG. 5, the pressing part 51 has a curved shape, which curls up, towards the back, along the circumference of a virtual circle P1 about a virtual center point S1. An erecting limb 52 is provided above the pressing part 51 along the forward and backward directions.

The forward projection part 60 is provided around the air hole 13, and projects forward from the center of the base part 10. The forward projection part 60 has an abutting part 61, an upper connecting part 62, a lower connecting part 63, an opposing wall part 64 and an opposing wall part 65.

The abutting part 61 is flat, and a penetrating hole 66 that penetrates in the direction of the plate width to communicate with the air hole 13 is provided (see FIG. 4). The abutting part 61 is provided to abut the base part 10.

The upper connecting part 62 is provided above the penetrating hole 66 and the air hole 13, facing the lower connecting part 63, and connects between the opposing wall part 64 and the abutting part 61. The upper connecting part 62 has a projecting part 67 that projects downward from the lower plane, which is the plane facing the lower connecting part 63, and that also projects in the air hole 13 and penetrating hole 66 when seen from the front.

The lower connecting part 63 is provided below the penetrating hole 66 and the air hole 13, facing the upper connecting part 62, and connects between the opposing wall part 65 and the abutting part 61. The lower connecting part 63 has a projecting part 68 that projects upward from the upper plane, which is the plane facing the upper connecting part 62, and that also projects in the air hole 13 and penetrating hole 66 when seen from the front. The projecting part 67 and the projecting part 68 are provided in opposing, upper and lower positions.

The opposing wall part 64, which is a third opposing wall part, is provided to extend upward from the front end of the upper connecting part 62, provides a gap, in which the upper lip can be inserted, between the opposing wall part 64 an the base part 10, and is provided to face the base part 10.

The opposing wall part 65, which is a fourth opposing wall part, is provided to extend downward from the front end of the lower connecting part 63, and provides a gap, in which the lower lip can be inserted, between the opposing wall part 65 an the base part 10, and is provided to face the base part 10. In the opposing wall part 65, as shown in FIG. 2, a penetrating hole 69 that penetrates in the direction of the plate width is provided. By connecting with a mounting tool (not shown) to mount on the head part and the jaws, the penetrating hole 69 connects the mounting tool and the oral cavity insertion tool 1 as one piece.

The middle part 80 connects between the lingual frenotomy pressing plate 50 and the forward projection part 60. In the middle part 80, an opposing wall part 81, which is a second opposing wall part to face the base part 10, which is provided to extend downward from the rear end, and which provides an interval, in which lower teeth can be inserted, between the opposing wall part 81 ad the base part 10, is provided. In the middle part 80, a dividing wall 82, which is provided farther in the back than the air hole 13 and the penetrating hole 66, and which is provided to divide the air hole 13 and the penetrating hole 66 into the upper part and the lower part seen from the front, is provided (see FIG. 2). The middle part 80 is provided to penetrate through the air hole 13 of the base part 10, and contained in the concave part 23. The rear support part 20 and the middle part 80 constitute a backward projection part.

In the lower plane of the dividing wall 82, a pair of projecting walls 83, which project downward, and which, seen from the front, project in the air hole 13 and the penetrating hole 66, are provided. As shown in FIG. 7, the pair of the projecting walls 83 are formed to part from each other, gradually, from the front towards the back, and are formed in the shape of the letter V, reversed and truncated, seen from below. Note that it is equally possible to form a single projecting wall 83 or form three or more. Also, the projecting walls 83 may be provided in an arrangement other than the shape of the letter V that is reversed and truncated when seen from below.

<Variation of Structure of Oral Cavity Insertion Tool>

A variation of the structure of the oral cavity insertion tool 100 according to an embodiment of the present invention will be described below, in detail, with reference to FIG. 8.

Parts in FIG. 8 that are structured the same as those of FIG. 1 to FIG. 6 will be assigned the same reference codes and their description will be omitted.

The plate width of the lower portion of the base part 101 where the abutting part 61 abuts is thicker than the plate width the lower portion of the base part 10 where the abutting part 61 abuts. Consequently, the distance L2 between the rear end of the base part 101 and the front end of the pressing piece 40 in the oral cavity insertion tool 100 is greater than the distance L1 between the rear plane of the base part 10 and the front end of the pressing piece 40 in the oral cavity insertion tool 1 (L2>L1) (see FIG. 4).

Note that components of the oral cavity insertion tool 100 other than the ones described above are structured the same as those of the oral cavity insertion tool 1, and therefore their description will be omitted.

<Method of Using Oral Cavity Insertion Tool>

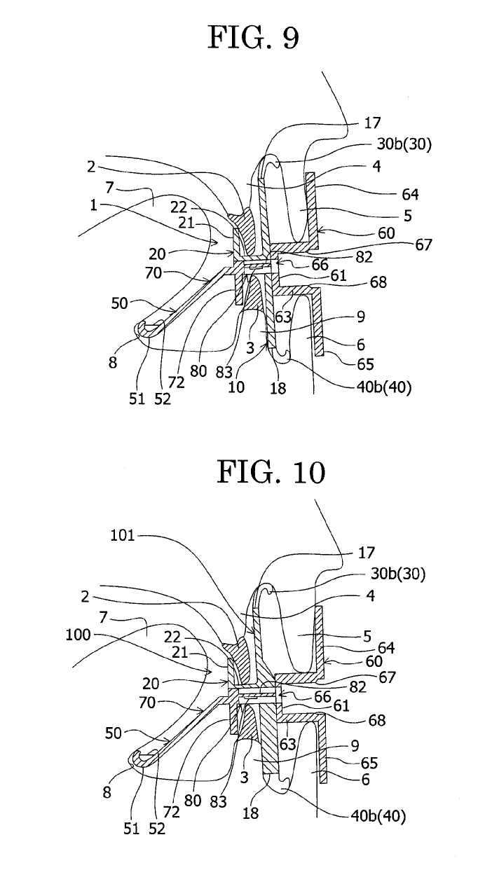

The method of using the oral cavity insertion tool 1 and the oral cavity insertion tool 100 according to an embodiment of the present invention will be described below, in detail, with reference to FIG. 9 and FIG. 10.

First, the user of the oral cavity insertion tool 1, when using the oral cavity insertion tool 1 during sleep and so on, as shown in FIG. 9, inserts the oral cavity insertion tool 1 in the oral cavity in the state the base part 10 abuts teeth and gums, inserts upper teeth 2 between the opposing wall part 21 and the base part 10, and inserts lower teeth between the opposing wall part 81 and the base part 10.

Also, the user of the oral cavity insertion tool 1 the upper lip 5 between the opposing wall part 64 and the base part 10, and also inserts the lower lip 6 between the opposing wall part 65 and the base part 10. By this means, the rear plane of the opposing wall part 64 abuts the exterior of the upper lip 5 and meanwhile the rear plane of the opposing wall part 65 abuts the exterior of the lower lip 6, preventing the oral cavity insertion tool 1 from moving into the oral cavity, so that the user of the oral cavity insertion tool 1 can prevent the situation where the whole of the oral cavity insertion tool 1 enters the oral cavity in unconscious states, typically during sleep and so on, and use the oral cavity insertion tool 1 safely.

Also, the pressing piece 30 is inserted between the gums 4 around the upper teeth 2 and the upper lip 5, and also the pressing piece 40 is inserted between the gums 9 around the lower teeth 3 side and the lower lip 6.

Furthermore, the pressing part 51 presses the lingual frenotomy 8 or the mucous membrane of the tongue base part to push the lingual frenotomy 8 downward and push the whole of the tongue 7 upward, thereby stimulating the lingual septum and rolling and moving the whole of the tongue 7 upward. In particular, the pressing part 51 rolls and moves upward the tongue root portion. By this means, it is possible to prevent constriction of the respiratory tract, which occurs because the tongue root is likely to sink in the respiratory tract.

The user of the oral cavity insertion tool 1, if necessary, connects the connecting tool that is connected to the mounting tool, which is mounted on the head part and the jaws, such as a string and/or the like, to the penetrating hole 69, and connects between the mounting piece and the oral cavity insertion tool 1. By this means, the user of the oral cavity insertion tool 1 can reliably keep the oral cavity insertion tool 1 placed in the oral cavity, even when using the oral cavity insertion tool 1 in unconscious states, typically during sleep and so on.

When women, young children and so on who exhibit a short distance from the mental spine to the lingual frenotomy 8 use the oral cavity insertion tool 1, given that the pressing part 51 is curved so as to curl up towards the back, the pressing part 51 is pressed forward by the lingual frenotomy 8 or the mucous membrane of the tongue base part, and deforms elastically to curl up further. By this means, the user of the oral cavity insertion tool 1 feels no pain when the lingual frenotomy pressing plate 50 deforms elastically. At this time, the hardness of the pressing part 51 improves by providing the limb 52 in the pressing part 51, so that it is possible to allow the pressing part 51 to deform elastically upward, smoothly.

On the other hand, as for adult males and so on who exhibit a long distance from the mental spine to the lingual frenotomy 8, the pressing part 51 abuts the lingual frenotomy 8 or the mucous membrane of the tongue base part, and the lingual frenotomy 8 or the mucous membrane of the tongue base part is reliably pressed by the pressing part 51. At this time, the hardness of the pressing part 51 improves by providing the limb 52 in the pressing part 51, so that it is possible to press the lingual frenotomy 8 or the mucous membrane of the tongue base part with the pressing part 51 reliably, with predetermined pressure force.

By providing the air hole 13 in the base part 10 and also providing the penetrating hole 66 in the abutting part 61, the user of the oral cavity insertion tool 1 is able to breathe through the air hole 13 and the penetrating hole 66 without feeling suffocated.

By providing the projecting part 67 that projects in the air hole 13 and the penetrating hole 66 seen from the front in the upper connecting part 62, and by also providing the projecting part 68 that projects in the air hole 13 and the penetrating hole 66 seen from the front in the lower connecting part 63, it is possible to make the flow of air drawn in from outside into the oral cavity via the penetrating hole 66 and the air hole 13, or the flow of air discharged to the outside from the oral cavity via the air hole 13 and the penetrating hole 66 turbulent. By this means, it is possible to make the flow rate of air drawn in from outside into the oral cavity via the penetrating hole 66 and the air hole 13, or the flow rate of air discharged to the outside from the oral cavity via the air hole 13 and the penetrating hole 66 fast, so that it is possible to ensure that the user of the oral cavity insertion tool 1 does not feel suffocated.

Also, by providing the dividing wall 82 to divide the air hole 13 and the penetrating hole 66 into the upper part and the lower part when seen from the front, and also by providing the projecting wall 83 that projects in the air hole 13 and the penetrating hole 66 when seen from the front in the dividing wall 82, it is possible to make the flow of air drawn in from outside into the oral cavity via the penetrating hole 66 and the air hole 13, or the flow of air discharged from the oral cavity to the outside via the air hole 13 and the penetrating hole 66 turbulent. By this means, it is possible to make the flow rate of air drawn in from outside into the oral cavity via the penetrating hole 66 and the air hole 13, or the flow rate of air discharged to the outside from the oral cavity via the air hole 13 and the penetrating hole 66 fast, so that it is possible to ensure that the user of the oral cavity insertion tool 1 does not feel suffocated.

Also, the pressing piece 30 is pressed against the upper lip 5 forward and the pressing piece 40 is pressed against the lower lip 6 forward. By trying to close the lips against the pressure force of the pressing piece 30 and the pressing piece 40, the user of the oral cavity insertion tool 1 can strengthen the surrounding of the orbicularis oris muscle. By constituting the pressing piece 30 with a plurality of the pressing piece 30a and the pressing piece 30b that are elastically deformable independently, and also by constituting the pressing piece 40 with a plurality of the pressing piece 40a and the pressing piece 40b that are elastically deformable independently, it is possible to apply load to the surrounding of the mouth depending on left and right, varying facial movements, so that it is possible to strengthen the orbicularis oris muscle in a good balance between the left and the right.

When reversed occlusion is desired, in which the lower jaw is placed ahead of the upper jaw, the oral cavity insertion tool 100 is used.

The oral cavity insertion tool 100 is made complete by removing the replacement member 70, which is attached to the base part 10 of the oral cavity insertion tool 1, from the base part 10, and attaching it to the base part 101.

To be more specific, by removing the middle part 80 from the air hole 13 of the base part 10 while pulling the replacement member 70 of the oral cavity insertion tool 1 forward and also elastically deforming the opposing wall part 72 backward, and by subsequently removing the lingual frenotomy pressing plate 50 from the air hole 13 of the base part 10 while pulling the replacement member 70 forward and also elastically deforming the lingual frenotomy pressing plate 50 backward, the replacement member 70 is removed from the base part 10. Then, the replacement member 70 is inserted in the air hole 13 of the base part 101 from the front while elastically deforming the lingual frenotomy pressing plate 50 backward, and subsequently the middle part 80 is inserted in the air hole 13 of the base part 101 from the front while elastically deforming the opposing wall part 72 backward, the replacement member 70 is inserted in the air hole 13 of the base part 101 from the front until the abutting part 61 abuts the base part 101. At this time, it is only necessary to insert the replacement member 70 in the air hole 13 until the abutting part 61 abuts the base part 10, it is possible to attach the replacement member 70 to the base part 101 reliably and easily. Note that the method of removing the replacement member 70 of the oral cavity insertion tool 100 from the base part 101 and attaching it to the base part 10 is the same as the above-described method, and their its description will be omitted.

In this way, it is possible to change the oral cavity insertion tool 1 to the oral cavity insertion tool 100 or change the oral cavity insertion tool 100 to the oral cavity insertion tool 1 by the simple process of switching the base part 10 and the base part 101, so that it is possible to assume non-reversed occlusion or reversed occlusion easily, by using the oral cavity insertion tool 1 or the oral cavity insertion tool 100.

The user of the oral cavity insertion tool 100 uses the oral cavity insertion tool 100 in which the distance L2 between the rear end of the base part 101 and the front end of the pressing piece 40 is greater than the distance L1 between the rear plane of the base part 10 and the front end of the pressing piece 40, and therefore can assume reversed occlusion by shifting the lower jaw forward and by pushing the lingual frenotomy 8 forward over a distance to match the distance the lower jaw is shifted forward. Note that the method of using the oral cavity insertion tool 100 is the same as the method of using the oral cavity insertion tool 1, and therefore its description will be omitted.

In this way, by using the oral cavity insertion tool 1 or the oral cavity insertion tool 100 depending on the distance from the mental spine to the lingual frenotomy, it is possible to prevent constriction of the respiratory tract, which occurs when the tongue root sinks in the respiratory tract, and reliably secure the respiratory tract.

According to the present embodiment, by pressing the lingual frenotomy or the mucous membrane of the tongue base part with the pressing part 51 that is curved so as to curl up towards the back at the rear end, it is possible to press the lingual frenotomy or the mucous membrane of the tongue base part reliably, without causing pain, and secure the respiratory tract, regardless of the distance from the mental spine to the lingual frenotomy.

The present invention is by no means limited to the above-described embodiment in terms of the types, the arrangement and the number of members, and can make changes as appropriate, without departing from the scope of the present invention, by, for example, adequately replacing these components with ones that bring about equivalent effects and advantages.

To be more specific, it is possible to provide a hook in the forward projection part 60 of the replacement member 70 and fix a string to the hook. The string fixed to the hook can be pulled forward when removing the oral cavity insertion tool 1 from the oral cavity. By this means, it is possible to remove the oral cavity insertion tool 1 from the oral cavity easily. Also, by pulling the string fixed to the hook in the state the upper lip and the lower lip are closed, upward, downward, to the left and to the right, it is possible to apply strong load to the muscles around the mouth and strengthen the orbicularis oris muscle even more.

Furthermore, although the forward projection part 60 is provided herein, it is equally possible not to provide the forward projection part 60. In this case, the lingual frenotomy pressing plate 50 and the middle part 80 are formed as one with the base part 10. By this means, it is possible to simply the structure of, and easily manufacture the oral cavity insertion tool 1.

Also, although the replacement member 70 is detachably attached to the base part 10 herein, it is equally possible to form the replacement member 70 and the base part 10 as one.

The disclosure of Japanese Patent Application No. 2015-113219, filed on Jun. 3, 2015, including the specification, drawings and abstract, is incorporated herein by reference in its entirety.

### INDUSTRIAL APPLICABILITY

The oral cavity insertion tool according to the present invention is suitable to insert in the oral cavity to secure the respiratory tract.

Reference Signs List

1 Oral cavity insertion tool

2 Upper teeth

3 Lower teeth

4 Gums

5 Upper lip

6 Lower lip

7 Tongue

8 Lingual frenotomy

9 Gums

10 Base part

11 Upper end

12 Lower end

17 Notch part

18 Notch part

20 Rear support part

21 Opposing wall part

22 Erecting part

30 Pressing piece

30a Pressing piece

30b Pressing piece

40 Pressing piece

40a Pressing piece

40b Pressing piece

50 Lingual frenotomy pressing plate

51 Pressing part

60 Forward projection part

61 Abutting part

62 Upper connecting part

63 Lower connecting part

64 Opposing wall part

65 Opposing wall part

67 Projecting part

68 Projecting part

69 penetrating hole

70 Replacement member

80 Middle part

81 Opposing wall part

82 Dividing wall

83 Projecting wall

1. An oral cavity insertion tool that is wholly elastically deformable and that is inserted in an oral cavity, the oral cavity insertion tool comprising:
a flat base part comprising:
a first pressing piece that is tabular and folded back forward at an upper end;
a second pressing piece that is tabular and folded back forward at a lower end and; and
an air hole that penetrates in forward and backward directions;
a backward projection part that projects backward from a center of the base part, and comprises:
a first opposing wall part that provides a gap in which upper teeth can be inserted between the first opposing wall part and the base part, and faces the base part; and
a second opposing wall part that provides a gap in which lower teeth can be inserted between the second opposing wall part and the base part, and faces the base part; and
a lingual frenotomy pressing plate that is tabular and provided to extend downward and backward from a rear end of the backward projection part,
wherein the lingual frenotomy pressing plate comprises a pressing part that curves to curl up, towards the back, at a rear end.

2. The oral cavity insertion tool according to claim 1, further comprising a forward projection part that projects forward from the center of the base part, and comprises:
a third opposing wall that provides a gap in which an upper lip can be inserted between the third opposing wall and the base part, and faces the base part; and
a fourth opposing wall that provides a gap in which a lower lip can be inserted between fourth opposing wall and the base part, and faces the base part.

3. The oral cavity insertion tool according to claim 2, wherein the forward projection part is provided around the air hole, and comprises a projecting part that projects in the air hole when seen from the front.

4. The oral cavity insertion tool according to claim 1, wherein the pressing part comprises an erecting limb that is provided above, along the forward and backward directions.

5. The oral cavity insertion tool according to claim 1, further comprising a dividing wall that is provided farther in the back than the air hole, and provided to divide the air hole when seen from the front,
wherein the dividing wall comprises a projecting wall that projects in the air hole when seen from the front.

6. The oral cavity insertion tool according to claim 1, further comprising a replacement member that has the second opposing wall part and the lingual frenotomy pressing plate, abuts the base part, and is detachably attached to the base part, the base part having a different plate width in a portion where the replacement member abuts.
